(19) United States
(12) Patent Application Publication
(10) Pub. No.: US 2011/0004089 A1
(43) Pub. Date: Jan. 6, 2011

(54) EAR-WORN EEG MONITORING DEVICE

(76) Inventor: Chang-An Chou, Taipei (TW)

Correspondence Address: Chang-An Chou, 3F, No. 100, Sec. 3. Mingsheng E. Rd., Taipei, 105, TW

(21) Appl. No.: 12/865,859

(22) PCT Filed: Feb. 5, 2009
(86) PCT No.: PCT/CN09/00133

§ 371 Date: Aug. 2, 2010

(30) Foreign Application Priority Data
Feb. 5, 2008 (CN) 200810080710.4

(51) Int. Cl. A61B 5/0478

(52) U.S. Cl. 600/383

(57) ABSTRACT

An ear-worn EEG monitoring device is disclosed. The EEG monitoring device includes plural electrodes, an EEG signal acquisition circuitry with RF module, at least a housing for accommodating the EEG signal acquisition circuitry with RF module, and at least an ear-worn structure, wherein the ear-worn structure can be implemented to mount on one or two ear(s) of the user, so that during the EEG monitoring process, the ear-worn structure can be used to hold and support the housing and the EEG signal acquisition circuitry therein at position(s) above and including the neck of the user, and the RF module is used for achieving a wireless communication with an external apparatus, so as to transmit acquired signals thereto.

### FIELD OF THE INVENTION

The present invention is related to an EEG (electroencephalograph) monitoring device, and more particularly to an ear-worn EEG monitoring device which provides convenient wireless operation with improved signal quality.

### BACKGROUND OF THE INVENTION

International 10-20 System is well adopted for EEG monitoring, which has become the international standard because of high reproducibility and accuracy. As illustrated in FIG. 1, this system divides the scalp into several parts. The longitudinal fiducial line passes through nasion and inion, and the overall distance is divided into six lengths: 10%, 20%, 20%, 20%, 20% and 10% with five points: Fpz, Fz, Cz, Pz and Oz from front to back. The transversal fiducial line passes through both pre-auricular points, and the overall distance is divided into six lengths: 10%, 20%, 20%, 20%, 20% and 10% with five points: T3, C3, Cz, C4 and T4 from left to right. The head circumference is the horizontal line passing through Fpz, T3, Oz and T4, wherein the left half distance is divided into six lengths: 10%, 20%, 20%, 20%, 20% and 10% with five points: Fp1, F7, T3, T5 and O1 from front to back, and the right half distance is divided into six lengths: 10%, 20%, 20%, 20%, 20% and 10% with five points: Fp2, F8, T4, T6 and O2 from front to back.

For general EEG monitoring, the wiring quantity and complexity for the electrodes attached to user's scalp is an important issue, for example, a basic 16 channels EEG monitoring requires 18 electrode wires, so that as more channels required, the electrode wiring quantity will be increased as well. Plus, traditionally, these electrode wires are connected to the EEG monitor aside the user, this kind of EEG monitor is disadvantageous that the electrode wires significantly restrict user's mobility, as well as the electrode wires are prone to be pulled to cause stability issue of signal acquisition. U.S. Pat. No. 5,479,934 discloses the similar situation (as shown in FIG. 14). This patent also discloses a headpiece for making EEG measurements on the head of a user which is constituted by a plurality of longitudinally extending and transversely extending elastic strips, and electrodes are assembled with the strips for fixing and positioning, and also, in this patent, part of the circuits and components can be hung on user's neck (as shown in FIG. 2), but electrode wires are still essential for outward connection to EEG monitor. Therefore, the problem of lacking of user mobility is still existed, and further, the module mount on user's neck may also make the user uncomfortable.

Moreover, U.S. Pat. No. 6,154,669 and USP Publication No. 2002/0188216 both disclosed of mounting EEG monitoring device on user's head by a mounting device (respectively the headset base and the headband). However, no matter the mounting device includes signal processing module therein or not, both EEG monitoring devices require connection wires (respectively cable wires 10A˜10D and headband cable 11) to connect to the external apparatus. Therefore, it still cannot provide sufficient mobility to the user, and the problem of wire pulling still exists. Moreover, when use headband to fix, as disclosed in USA Patent Application 2002/0188216, the headband may occupy the attaching position of EEG electrode, and also cause inconvenience for electrode wiring. These make the monitoring becomes difficult accordingly.

Furthermore, the portable EEG monitoring device is developed, such as AURA24 Ambulatory EEG System manufactured by Grass Technologies. The main device of this system is designed to be carried on the body for providing the user a better mobility. However, no matter the main device is carried on the chest or tied on the waist, it still brings inconvenience. First, though the device volume is reduced to a portable size, to carry the device on the body is not a natural way, so that it is still a burden to the user. Second, though the device does not require connecting to other external apparatus, the electrode wires are still prone to be pulled since the wires connected from head to chest or waist might go around the body. And, more importantly, this kind of EEG monitoring system still needs to be connected to the monitor aside the body as performing real-time monitoring, so a considerable limitation to the mobility still exists.

Other examples are the AirEEG manufactured by NIHON KOHDEN and Siesta manufactured by Compumedics, both of which are provided with wireless transmission interface, so acquired EEG signals can be transmitted to the monitoring device within the RF range. Thus, high user mobility and real-time monitoring can be achieved at the same time. However, the major problems of this kind of EEG monitoring device still are the burden caused from unfamiliar carrying manner and easily-pulled electrode wires.

As can be seen, no matter the traditional EEG monitoring device or the portable version, the situation of unstable signal acquisition caused by wire pulling is still unable to be avoided.

Besides, because EEG signals are very weak, how to acquire clear EEG signals has become the research focus for a long time. However, as well known by skills in the art, longer electrode wire produces more resultant noises in the signal. Therefore, configuration of electrode wiring is also a very important task in EEG monitoring.

In addition, since many diseases, such as, sleep disorders, brain trauma, or other brain diseases, e.g., Alzheimer's disease, schizophrenia, etc. should be diagnosed by EEG monitoring device, EEG monitoring is getting popular and more attention has been brought thereto. Thus, the using practice also should be considered as designing an EEG monitoring device, which has already become the key point of research, in addition to shortening electrode wires.

Therefore, an object of the present invention is to provide an ear-worn EEG monitoring device, which selects user's ear(s) as the disposing position, so that not only the lengths of electrode wires can be reduced significantly, the possibility of wire pulling is also decreased, thereby both better signal quality and improved diagnostic accuracy can be achieved.

Another object of the present invention is to provide an ear-worn EEG monitoring device, which utilizes user's ear(s) as holding and supporting medium for providing a using practice closer to a regular earphone, so as to alleviate the inconvenience caused by using the other monitoring device.

Further object of the present invention is to provide a wireless ear-worn EEG monitoring device, which brings higher mobility to the user, as well as satisfies the requirement for real-time monitoring.

### SUMMARY OF THE INVENTION

In one aspect of the present invention, an ear-worn EEG monitoring device is provided. The device includes plural electrodes, an EEG signal acquisition circuitry with RF module, a housing for accommodating the EEG signal acquisition circuitry, and an ear-worn structure mounted on one single ear of the user, wherein during the EEG monitoring process, the ear-worn structure is used to hold and support the housing and the EEG signal acquisition circuitry therein at a position around said ear, and the RF module is used for achieving a wireless communication with an external apparatus, so as to transmit acquired signals thereto.

In a preferred embodiment, two ear-worn structures and two housings are provided, and each housing is respectively attached with one single ear-worn structure, and further, the EEG signal acquisition circuitry is separated into two electrically connected modules for locating in two housings. Alternatively, in another preferred embodiment, the housing can be formed to have a special shape to connect with both ear-worn structures.

In a second aspect of the present invention, the ear-worn structure can be implemented to mount on both the user's ears, so that during EEG monitoring process, the dual-ear-worn structure can be used to hold and support the housing and the EEG signal acquisition circuitry therein to locate at a position above and including the neck of the user.

According to the description above, it is preferable that the housing is positioned around one of the user's ears or at the nape of the user's neck, so as to obtain a better support.

In a third aspect of the present invention, the number of housing is implemented to be multiple, and accordingly, the EEG signal acquisition circuitry with a RF module is separated into multiple modules with electrical connection therebetween for disposing in multiple housings, wherein multiple housings are attached to and distributed over the ear-worn structure. During the EEG monitoring process, the ear-worn structure is used to hold and support multiple housings and the EEG signal acquisition circuitry therein at positions above and including the neck of the user.

In a preferred embodiment, multiple housings can be selected to place to the following positions, including, but not limited, both the user's ears, the nape of user's neck, the top of user's head, and user's forehead. However, it should be noticed that there is no limitation to the combination between the ear-worn structure and multiple housings, and the disposing positions for the housings can be varied for different requirements.

Preferably, in view of above, for reducing the wiring complexity, the electrical connection between modules can be located in the ear-worn structure, and/or a holding element, which is used to connect to both housings to assist in holding and supporting the ear-worn structure, and/or an electrode-positioning element, which is used to locate the position of electrode(s).

Advantageously, the ear-worn structure can be integrated with at least one electrode, such as a reference electrode for disposed on user's ear, or mastoid. Moreover, the electrode integrated with the ear-worn structure also can be electrodes other than those located around the ear(s), that is, electrode positions located on the way the ear-worn structure passing through can be implemented to integrate therewith.

It is further advantageous that a memory can be further included in the ear-worn EEG monitoring device for data storage, and in a preferred embodiment, the memory can be implemented as a removable memory for external access, for example, the user can just takes the memory rather than the whole device to visit the doctor for interpreting the result.

Besides, through the wireless communication with the external apparatus, e.g., PC, acquired EEG signals can have a real-time transmission to the external apparatus, so that the requirement of real-time monitoring, which is important for EEG research, also can be achieved.

As to the external apparatus, it is used for monitor, analysis and/or storage during and after the EEG monitoring process, for example, it can be a computer for performing EEG signal processing or an EEG signal recorder with wireless input interface, and through the RF module, the external apparatus can control of the EEG monitoring device, such as, configuring settings and parameters. And, if the external apparatus is provided with the function of networking, the real-time monitoring can even be monitored by remote medical personnel.

### BRIEF DESCRIPTION OF THE DRAWINGS

A more detailed understanding of the invention may be had from the following description of a preferred embodiment, given by way of example, and to be understood in conjunction with the accompanying drawings, wherein:

FIG. 1 is a schematic view showing the international 10-20 system for EEG monitoring;

FIGS. 2A˜2B are schematic views showing two using examples of the ear-worn EEG monitoring device according to the present invention;

FIGS. 3A˜3B are schematic views showing the exemplary structures of the ear-worn EEG monitoring device according to the present invention;

FIGS. 4A˜4C are schematic views showing the application examples of the ear-worn EEG monitoring device according to the present invention;

FIG. 5 is a circuit block showing the EEG signal acquisition circuitry with RF module and electrodes according to the present invention

FIGS. 6A˜6C are schematic views showing the exemplary circuit arrangements of the EEG signal acquisition circuitry with electrodes according to the present invention;

FIGS. 7A˜7B are schematic views showing the exemplary electrode arrangements according to the present invention;

FIG. 8 is a schematic view showing an exemplary electrode structure according to the present invention; and

FIG. 9 is a schematic view showing an exemplary application of the electrode-positioning element according to the present invention.

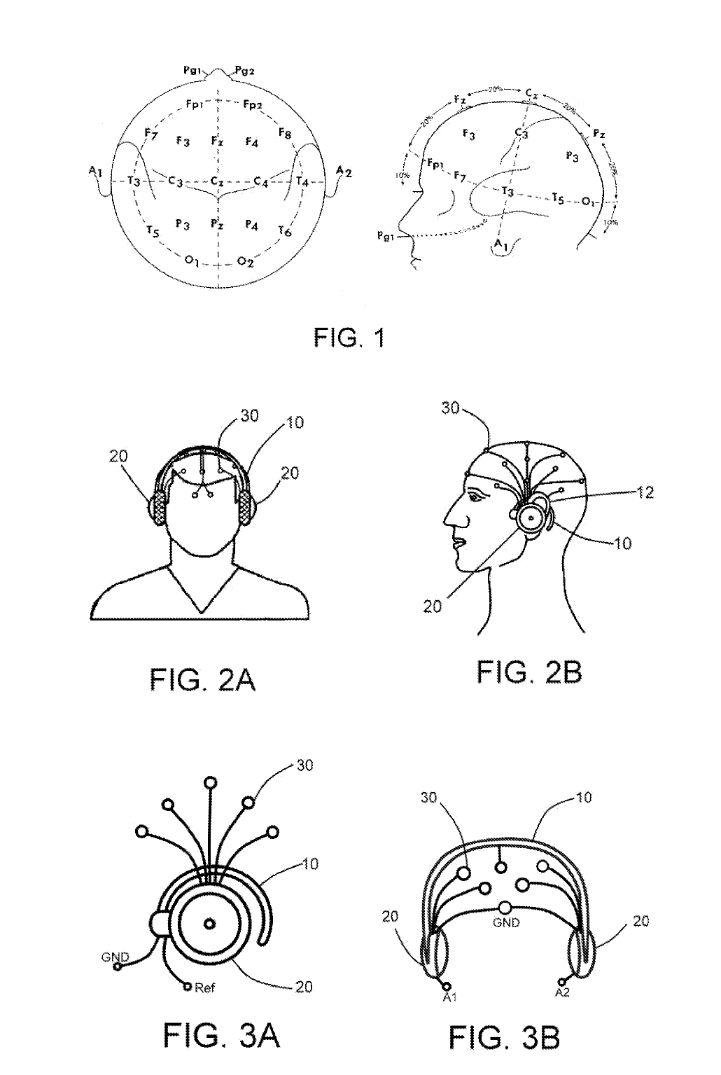

### DETAILED DESCRIPTION OF THE PREFERRED EMBODIMENTS

Generally, there are two major ways to perform EEG monitoring. One is to employ electrode wires from user to EEG monitor aside user's body, which restricts user's movement seriously, and another kind of EEG monitor is designed to be portable, which might be inconvenient to users due to loading and carrying issue. Accordingly, the object of the present invention is to provide a novel configuration of EEG monitor which provides ergonomic design for reducing installation complexity and simplifying operation procedure.

The present invention is related to an ear-worn monitoring device, which improves the conventional hardware configuration and complicated electrode wiring for providing convenient installations of device and electrodes, so as to increase user's acceptance of EEG monitoring and diagnosis.

Please refer to FIGS. 2A˜2B, which are schematic views respectively showing the applications of the ear-worn EEG monitoring device according to the present invention in two different situations. As shown, the ear-worn EEG monitoring device includes an ear-worn structure 10 to hold and support housing(s) 20 on user's ear(s) 12. The reasons why ear(s) is selected to be the holding medium are:

1. Since EEG monitoring disposes electrodes on user's scalp for acquiring EEG signals, it is a natural choice to close the EEG monitoring device to user's head. That's because the closer the position of the EEG monitoring device, the shorter the length of the electrode wires, so that the electrode wires can be simplified to locate only around the head, and user's mobility and operation convenience can have significant improvement. More importantly, this configuration can reduce the possibility that the electrode(s) might be pulled to influence the contact condition with the scalp by movements of other body portions, such as, hands, or by objects around the monitoring environment, such as, table or chair, so that the reliability of signal acquisition can accordingly be improved.

2. The structure of ears is suitable for hanging or mounting objects thereon, and many electronic products, e.g., MP3 Player, Bluetooth earphone, have already employed this mounting mechanism, so it is very familiar and also acceptant for the public to mount object on ear(s).

3. It is well known that EEG monitoring acquires signals from areas around ears, such as, earlobe and mastoid, by disposing electrodes therearound, so that it is natural to integrate the electrode with the ear-worn structure for reducing number of electrode wires and thus simplifying electrode installation.

Therefore, according to the reasons described above, the present invention employs the ear-worn structure to hold and support the EEG monitoring device.

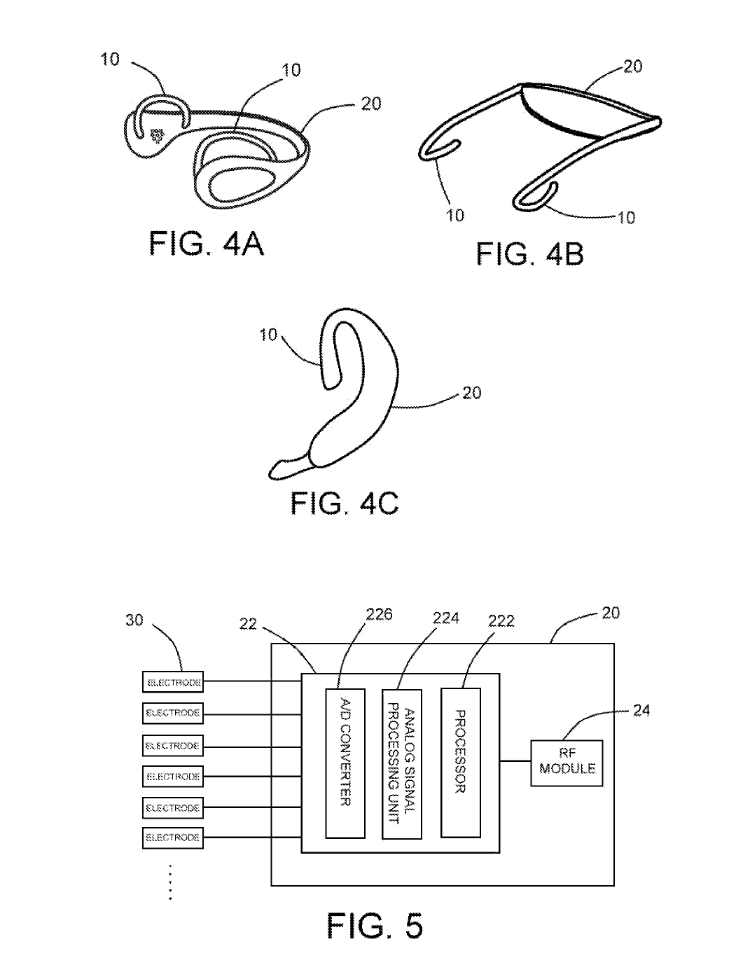

Then, please refer to FIGS. 3A˜3B, which are schematic views respectively showing the ear-worn EEG monitoring devices in preferred embodiments of the present invention. As shown in FIG. 3A, the EEG monitoring device includes an ear-worn structure 10, a housing 20 for accommodating EEG signal acquisition circuitry (not shown, described below) therein, and plural electrodes 30 connected to the EEG signal acquisition circuitry for signal acquisition. Here, the ear-worn structure 10 is mounted on one of user's ears, and the housing 20 is supported by the ear-worn structure 10 for locating around the ear. As shown in FIG. 3B, two housings 20 are mounted on both ears by one ear-worn structure 10, and plural electrodes 30 are extended from the housings 20. Here, it should be noticed that, without limitation, the number of the housing 20 can be varied according to different monitoring demands, and further, the position of the housing(s) on the ear-worn structure also can be different, for example, FIGS. 4A˜4C show the exemplarily different choices. That is, in the present invention, for corresponding to circuit scale, operation convenience and electrode arrangement, the EEG monitoring device can be implemented to have one or more housings with flexible fixing position on different types of ear-worn structures.

For example, as the housing is held and supported by one single-ear-worn structure, in addition to the example shown in FIG. 3A, the single housing of the EEG monitoring device also can be located behind the hear, as shown in FIG. 4C, or other positions where the holding and supporting of the housing can be achieved by the single-ear-worn type structure. Further, the implementation of single-ear-worn structure with single housing can be expanded to be two single-ear-worn structures with two housings, and thus, the EEG signal acquisition circuitry can be divided into two electrically connected modules for respectively locating in two housings, just like the common clip-on earphone with housing connecting line, thereby distributing the burden on the user. Or, more specially, two single-ear-worn structures can be used to hold and support one single housing, as shown in FIG. 4A. Here, the housing 20 is formed to have a special shape for connecting to both single-ear-worn structures, and the EEG signal acquisition circuitry can be distributed in the extended housing.

Alternatively, the ear-worn structure also can be implemented to mount on both the user's ears, just like the examples shown in FIG. 3B and FIG. 4B. As using the dual-ear-worn structure, the position of housing may even have more choices. For example, FIG. 3B shows the situation of two housings respectively located on two ears, and the ear-worn structure crosses the top of the head, and FIG. 4B shows an embodiment that one single housing is attached to the dual-ear-worn structure and located at the nape of the neck. But, it should be noticed that these are only exemplary illustrations and the present invention is not restricted thereby. For example, it also can be implemented to have two or more housings distributed on the dual-ear-worn structure shown in FIG. 4B, as well as the positions for disposing housings can be, e.g., both ears, the nape of neck and/or any section of the dual-ear-worn structure only if the holding and supporting functions can be kept. Also, the same situation can be applied to the dual-ear-worn structure shown in FIG. 3B for increasing the number of housing, for example, a housing located at the top of head. In addition to two housings located on both ears, housing(s) to locate at other positions of the dual-ear-worn structure is also applicable.

Besides, for simplifying the electrode wires, it is better to locate the housing(s) at position(s) above (including) user's neck. Accordingly, through providing electrodes wires with proper length, the electrode wires will only go around user's head and will not extend along the body, so that the possibility of accidental pulling to electrode wires can be minimized, and further, the user can freely turn his/her head and the limitation to normal limb movements, such as, hands, also can be reduced.

Therefore, the ear-worn structure(s) and the housing(s) of the EEG monitoring device according to the present invention can have various combinations only if the housing(s) can be held and supported by the ear-worn structure(s) to position above (including) the neck. Additionally, for achieving better holding and supporting capabilities, a holding element (not shown) also can be further provided for connecting to the housing(s), there is no limitation.

Now, please refer to FIG. 5, which is a circuit block showing the EEG signal acquisition circuitry of the present invention. The EEG signal acquisition circuitry 22 includes a processor 222, an analog signal processing unit 224, and an A/D (analog/digital) converter 226, but not limited, for example, filter and amplifier also can be included therein.

Further, as shown, the EEG signal acquisition circuitry 22 also includes a RF module 24 for performing wireless communication. In addition to off-line analysis of EEG signals, real-time monitoring is also an important category in brain research, so that for complying with this requirement, the ear-worn EEG monitoring device of the present invention further employs the RF module 24 to perform wireless signal transmission to external apparatus, such as, computer, with compatible communication interface. Moreover, based on the RF module 24, the external apparatus also can wirelessly control and configure the ear-worn EEG monitoring device, for example, start/stop operation and parameter settings, that is, the EEG signal acquisition device can have a wireless communication with the external apparatus, even during the monitoring process. Besides, since one purpose of the present invention is to maximize user mobility during EEG monitoring, it will be better to eliminate the connecting cable between the EEG monitoring device and the external apparatus, just like the cable 11 in USP Publication No. 2002/0188216 (which is adapted to connect to the recorder 30), thereby the user mobility won't be sacrificed by real-time monitoring. Therefore, through employing the RF module 24, the reduction of wiring complexity, the enhancement of user mobility, and multiple monitoring benefits all can be achieved. Here, it should be noticed that there is no limitation to the circuit arrangement (electric components and/or modules) between the ear-worn EEG monitoring device and the external apparatus, which means the functions provided by the ear-worn EEG monitoring device is flexible, for example, the acquired EEG signals can be directly transmitted to the external apparatus after digitization, or can be processed before transmission, various situations are possible.

Although the ear-worn EEG monitoring device of the present invention is provided with the RF module, a memory is also applicable. For example, the memory can be used to store EEG signals during the entire monitoring process, no matter the wireless real-time transmission is executed or not, for off-line analysis purpose; or the memory can be used as the buffer during wireless transmission, so that when the user is out of the receiving range of the external apparatus, the signals still can be temporarily stored for future transmission as the user is back into the receiving range; or the memory can be used to store a backup in case of poor signal quality of wireless transmission. Plus, for outputting the data stored in the memory, the ear-worn EEG monitoring device can be provided with a wired transmission interface in addition to the original RF module, such as, USB and 1394. Alternatively, the memory also can be implemented to be removable, so that data access can be executed outside the EEG monitoring device.

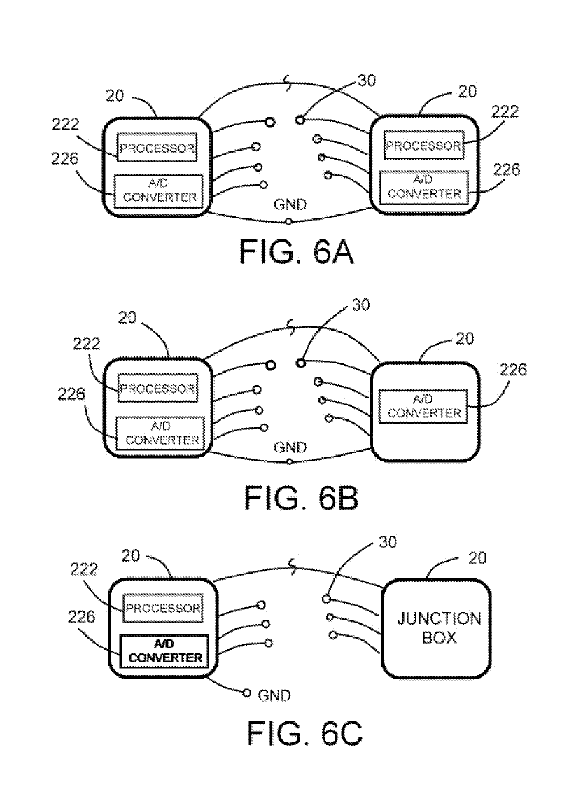

Furthermore, when the number of the housing is implemented to be multiple, the circuit arrangement for the EEG signal acquisition circuitry can be varied. For example, FIGS. 6A˜6C show exemplary circuit arrangements of the EEG signal acquisition circuitry according to the present invention. First of all, it should be noticed that in FIGS. 6A˜6C, for clarity, only circuit components involved in description are shown, which doesn't mean to limit the present invention. As shown in FIG. 6A, both housings 20 include processor 222 and A/D converter 226, so that two processors 222 can have a communication (including exchanging digitized signals) through the electrical connection therebetween. In this embodiment, electrodes 30 can be extended from modules in both housings without limitation, but as known, both modules should be connected to one GND, as shown. FIG. 6B shows the situation that only one housing is provided with processors 222 and both housings include A/D converter 226, so that the processor 222 can control the module in the other housing through the electrical connection therebetween. FIG. 6C shows the situation that one housing 20 is implemented to have a function of junction box. The junction box is responsible for connecting with electrodes nearby and collecting and transmitting signals to the housing 20 having processor 222 and the A/D converter 226 therein. In this embodiment, the collected signals can be directly transmitted in analog format, and then digitized and processed in the other housing 20. Of course, digitization before transmission is also possible. This is especially suitable for EEG monitoring which needs to employ large amount of electrodes, so that the length of electrode wires can be effectively reduced, and so to the manufacturing cost. Here, the electrical connection can be accommodated in the ear-worn structure and/or the holding element for reducing the wiring complexity.

Of course, these are only described for illustration, not for limitation. There still are many other choices, no matter in circuit arrangement or in hardware configuration. For example, the number of housing can be more than two, such as, three, and one housing can be used for accommodating battery only, so that the power will not be limited by volume.

As to electrodes, the amount and arrangement thereof both can be varied corresponding to different demands. For example, when being used in general EEG monitoring, it is common to use 16 channels, 32 channels or 64 channels acquisition; or when being used for approximately realizing the variation of brain waves only, the needed amount of electrodes becomes fewer, such as, typically, C3, C4, O1, O2 signals are needed in sleep study. Thus, there is no limitation.

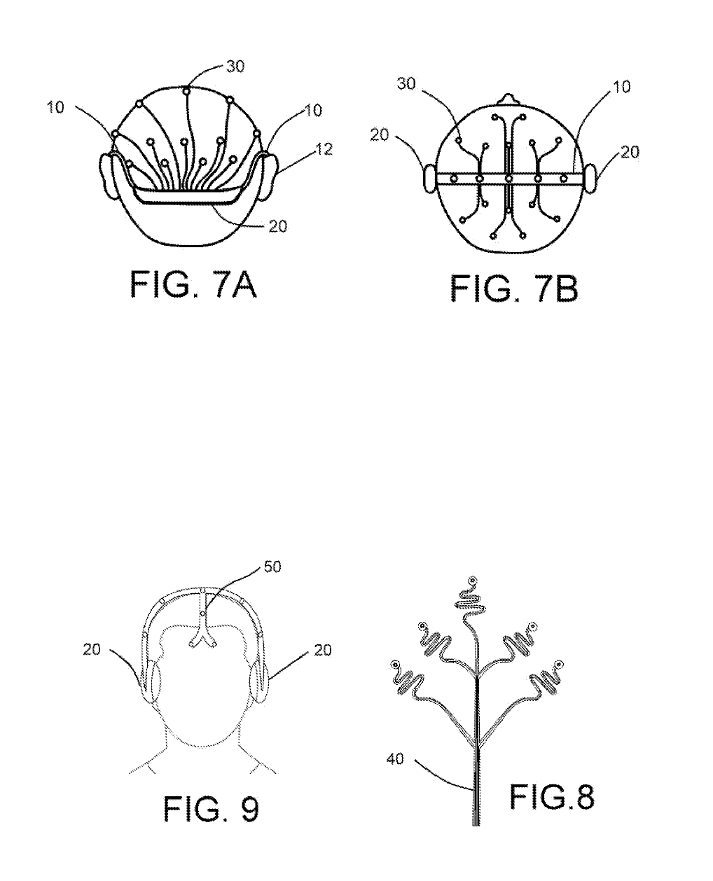

Moreover, the electrodes also can be integrated with the ear-worn structure. For example, the electrode(s) integrated with the ear-worn structure can be used to acquire signals from references A1, A2, or positions around the ears, such as mastoid. Plus, according to different types of ear-worn structures, electrodes at other signal acquisition positions also can be integrated with the ear-worn structure. For example, the ear-worn structure in FIGS. 2A and 3B can be integrated with electrodes located in the transversal fiducial line, as shown in FIG. 7A, and the ear-worn structures in FIGS. 4A and 4B can be integrated with O1, O2 electrodes, and even T5, T6, T3 and T4 electrodes.

Furthermore, the arrangement of electrode wires also can be varied in accordance with different hardware configurations of the EEG monitoring device. In addition to the conventional way, as shown in FIG. 7A, that one electrode employs one connecting wire extended from the housing, it also can be, as shown in FIG. 7B, the ear-worn structure which crosses the top of the head accommodates parts of the electrode wires from the housings and provides output sites at proper positions. Or, all electrode wires can be firstly collected in one bundle for extending from the housing, and then gradually separated into more electrode wires as the bundle arriving respective electrode positions. Therefore, the arrangement of electrode wires is depending on hardware configuration (the number and position of housing and the type of ear-worn structure), and can be varied accordingly.

Particularly, FPCB (flexible printed circuit board) is also a suitable form for carrying electrode wires. FPCB is featured of flexible and capable of mounting electronic components, so that the functions of carrying electrode wires and fitting head's curve can be achieved at the same time, and further, because FPCB is also characteristic of lightweight, the FPCB-carried electrode wires can effectively reduce the weight added on user's head as compared with the traditional electrode wires. FIG. 8 illustrates one kind of electrode FPCB, which starts from gathered multiple wires and gradually separates into respective electrode wires, and even, as shown, through designing the shape of FPCB, an extendable function can be obtained additionally. Moreover, a further advantage can be obtained from FPCB-carried electrode wires, that is, since the electrode wires are integrated on FPCB, the movement of user will not change the relative distance among electrode wires, so that the interference between traditional electrode wires and the noises produced therefrom can be minimized.

Furthermore, as shown in FIG. 9, an electrode-positioning element 50 can be provided for assisting in electrode locating and fixing. The electrode-positioning element 50 which crosses the ear-worn structure at point Cz can provide the position indications of Fz, Pz and Fp1, Fp2, O1, O2. Here, the electrode wires can be implemented to accommodate therein for reducing complexity, and the length of the electrode-positioning element can be implemented to be adjustable for adapting to different users. Therefore, through the design shown in FIG. 9, when the user wears the EEG signal monitoring device, it only needs to make sure that two housings are located on the ears and the crossing point, Cz, is positioned at the center of head top, then electrode positions at the longitudinal fiducial line and the transversal fiducial line are decided, and further, electrodes for F7, F3, F4, F8 and T5, T3, P4, T6 can be respectively extended from T3, C3, C4, T4 (for example, the lengths of extended electrode wires can be limited for ensuring the right positioning of electrodes). Of course, the implementation shown in FIG. 9 is only for illustration, not for limitation, and other implementations are also possible, for example, the electrode-positioning element can be purely used for locating the positions of electrodes and not accommodating electrode wires and/or combining with electrodes, so that after the electrode positions are confirmed, the electrode-positioning element will be removed; or the electrode-positioning element also can be integrated with the holding-element.

Besides, based on an impedance check provided in the ear-worn EEG monitoring device of the present invention, the user can confirm if the contact between electrode and scalp is well enough.

Accordingly, according to the present invention, of EEG monitoring can have a simpler operation procedure, and thus, the EEG monitoring can be applied to more situations, such as, biofeedback, brain training, and ERP (Event-Related Potential) testing (e.g., ERP 300), so as to benefiting more people.

In the aforesaid, the ear-worn EEG monitoring device according to the present invention utilizes ear(s) as the medium for holding and supporting the whole device, so that not only the lengths of electrode wires can be significantly reduce, the wiring complexity also can be simplified, thereby the noise level caused by electromagnetic interference can be minimized. Moreover, the ear-worn design provides user a familiar using style, just like wearing an earphone, or headphone, so that user won't feel burden or uncomfortable. Plus, the equipped RF module, in addition to highly improves user's mobility during monitoring process, also achieves the requirement of real-time monitoring. Consequently, the ear-worn EEG monitoring device of the present invention is not only advantageous of electromagnetic interference reduction and accuracy improvement, but also beneficial to provide more familiar using practice which broadens the application scope and also increases user's acceptance.

What is claimed is:

1. An ear-worn EEG monitoring device, comprising:
plural electrodes;
an EEG signal acquisition circuitry with RF module;
a housing, for accommodating the EEG signal acquisition circuitry; and
an ear-worn structure, mounted on one single ear of the user;
wherein during the EEG monitoring process,
the ear-worn structure is used to hold and support the housing and the EEG signal acquisition circuitry therein at a position around said ear, and
the RF module is used for achieving a wireless communication with an external apparatus, so as to transmit acquired signals thereto.

2. The monitoring device as claimed in claim 1, wherein the housing is positioned behind the ear.

3. The monitoring device as claimed in claim 1, wherein the number of the ear-worn structure is implemented to be two.

4. The monitoring device as claimed in claim 3, wherein the number of the housing is implemented as two, and each housing is respectively attached with one single ear-worn structure.

5. The monitoring device as claimed in claim 4, wherein the EEG signal acquisition circuitry is separated into two electrically connected modules for locating in two housings.

6. The monitoring device as claimed in claim 3, wherein the housing is formed to connect with both single ear-worn structures.

7. The monitoring device as claimed in claim 1, wherein the ear-worn structure is integrated with at least one of the electrodes.

8. The monitoring device as claimed in claim 7, wherein the electrode integrated with the ear-worn structure is disposed on the ear.

9. The monitoring device as claimed in claim 7, wherein the electrode integrated with the ear-worn structure is disposed on the mastoid.

10. The monitoring device as claimed in claim 7, wherein the electrode integrated with the ear-worn structure is a reference electrode.

11. The monitoring device as claimed in claim 1, further comprising an electrode-positioning element, for fixing the electrodes.

12. The monitoring device as claimed in claim 1, wherein the external apparatus is used for monitor, analysis and/or storage.

13. The monitoring device as claimed in claim 1, wherein based on the RF module, the external apparatus configures the EEG monitoring device.

14. The monitoring device as claimed in claim 1, wherein the ear-worn EEG monitoring device further comprises a memory for storing EEG signals.

15. The monitoring device as claimed in claim 14, wherein the memory is a removable memory.

16. The monitoring device as claimed in claim 1, wherein the ear-worn EEG monitoring device further comprises a wired communication interface for communicating with the external apparatus.

17. An ear-worn EEG monitoring device, comprising:
plural electrodes;
an EEG signal acquisition circuitry with a RF module;
a housing, for accommodating the EEG signal acquisition circuitry; and
an ear-worn structure, mounted on both the user's ears, wherein
during the EEG monitoring process,
the ear-worn structure is used to hold and support the housing and the EEG signal acquisition circuitry therein to locate at a position above and including the neck of the user; and
the RF module is used for achieving a wireless communication with an external apparatus, so as to transmit acquired signals thereto.

18. The monitoring device as claimed in claim 17, wherein the ear-worn structure is integrated with at least one of the electrodes.

19. The monitoring device as claimed in claim 17, wherein the housing is positioned around one of the user's ears or at the nape of the user's neck.

20. An ear-worn EEG monitoring device, comprising:
multiple housings;
an EEG signal acquisition circuitry with a RF module, separated into multiple modules with electrical connection therebetween for disposing in multiple housings;
plural electrodes; and
an ear-worn structure, mounted on both the user's ear,
wherein
multiple housings are attached to and distributed over the ear-worn structure; and
during the EEG monitoring process,
the ear-worn structure is used to hold and support multiple housings and the EEG signal acquisition circuitry therein at positions above and including the neck of the user, and
the RF module is used for achieving a wireless communication with an external apparatus, so as to transmit acquired signals thereto.

21. The monitoring device as claimed in claim 20, wherein multiple housings are at positions at least two selected from both the user's ears, the nape of user's neck, the top of user's head, and user's forehead.

22. The monitoring device as claimed in claim 20, wherein the electrical connection is located in the ear-worn structure.

23. The monitoring device as claimed in claim 20, wherein the monitoring device further comprises a holding element connecting to both housings for assisting the holding and supporting function of the ear-worn structure.

24. The monitoring device as claimed in claim 20, wherein the electrical connection is located in the holding element.
